(19) United States
(12) Patent Application Publication
(10) Pub. No.: US 2022/0387646 A1
(43) Pub. Date: Dec. 8, 2022

(54) DUAL ZONE SYSTEMS AND METHODS FOR DISINFECTING WITH OZONE

(71) Applicant: Preservation Tech LLC, Hull, IA (US)

(72) Inventors: Kevin York, Joplin, MO (US); Sannel Patel, Hull, IA (US)

(21) Appl. No.: 17/831,038

(22) Filed: Jun. 2, 2022

Related U.S. Application Data
(60) Provisional application No. 63/196,035, filed on Jun. 2, 2021.

(51) Int. Cl. A61L 2/20; C01B 13/11

(52) U.S. Cl. CPC A61L 2/202; C01B 13/11; A61L 2202/11; A61L 2202/122; A61L 2202/14

(57) ABSTRACT

An ozone conveyor includes a conveyor belt extending through an elongated enclosure. The conveyor also includes an aqueous ozone section with an aqueous ozone applicator mounted next to the conveyor belt and a gaseous ozone section with a gaseous ozone applicator mounted next to the conveyor belt. One or more exhaust ports are configured to connect to an exhaust system for removing gaseous ozone from the enclosure. An ozone disinfection system includes a dual zone ozone generator and an exhaust system operably coupled with an ozone conveyor. The dual zone ozone generator includes an ozone generator that produces gaseous ozone and an ozone injector for producing aqueous ozone. A method for disinfecting with ozone includes applying aqueous and gaseous ozone to a product and maintaining a net negative air pressure to reduce the likelihood of ozonated air leaks.

### CROSS-REFERENCE TO RELATED APPLICATION(S)

This application claims the benefit under 35 U.S.C. § 119(e) to U.S. Provisional Application 63/196,035, filed Jun. 2, 2021, and entitled “Dual Zone Systems and Methods for Disinfecting Meat Products,” and to U.S. Provisional Application 63/282,602, filed Nov. 23, 2021, and entitled “Dual Zone Systems and Methods for Disinfecting with Ozone,” both of which are hereby incorporated herein by reference in their entireties.

### TECHNICAL FIELD

The disclosed technology relates generally to systems and method for disinfecting products including, for example, food products and meat products, with ozone.

### BACKGROUND

As poultry processing facilities are continually looking for alternative technologies that balance cost versus result, further evaluation of different biocides having different molecular structures and different modes of action has become of significant interest. Alternative disinfection methods have become a part of any corporation's social responsibility plan to lower the environmental impact that harsh antimicrobials potential pose. In addition, the production of safe, healthy food requires that organizations pursue alternative means to lower the risk of food borne organisms while providing a safe environment for their employees. Peracetic acid, “PAA,” has become a reliable yet versatile antimicrobial in the defense of safe food production; however, more tools are needed that provide an added layer of protection in lowering the risk of naturally occurring pathogenic organisms. Thus, there is a need in the art for improved biocides, that can be applied safely and economically.

### SUMMARY

This disclosure generally relates to disinfecting products with ozone. Various aspects of the disclosure relate to ozone conveyors and disinfection systems, along with methods for disinfecting with ozone.

An ozone conveyor is provided according to one aspect of the disclosure. The conveyor includes an elongated enclosure, a conveyor belt extending through the enclosure, an aqueous ozone section, a gaseous ozone section, and one or more exhaust ports. The aqueous ozone section includes an aqueous ozone applicator mounted next to the conveyor belt. The gaseous ozone section includes a gaseous ozone applicator mounted next to the conveyor belt. The exhaust port(s) are configured to connect to an exhaust system for removing gaseous ozone from the enclosure.

Various implementations of the ozone conveyor include one or more of the following features and/or elements. In some cases the elongated enclosure includes an entrance at a first end of the enclosure and an exit at a second end of the enclosure. In such cases, the aqueous ozone section includes a first portion of the enclosure between the entrance and the exit and the gaseous ozone section includes a second portion of the enclosure between the first portion of the enclosure and the exit. In various implementations the ozone conveyor further includes an exhaust hood mounted at the exit of the enclosure that includes the one or more exhaust ports. In various implementations the aqueous ozone applicator is a first aqueous ozone applicator mounted above the conveyor belt. The aqueous ozone section further includes a second aqueous ozone applicator mounted below the conveyor belt. In various implementations the gaseous ozone applicator is a first gaseous ozone applicator mounted above the conveyor belt. The gaseous ozone section further includes a second gaseous ozone applicator mounted below the conveyor belt.

In various implementations the aqueous ozone applicator includes a first plurality of nozzles coupled to a first spray manifold in a first configuration and the gaseous ozone applicator includes a second plurality of nozzles coupled to a second spray manifold in a second configuration. In some cases the spacing of the first plurality of nozzles in the first configuration is denser than the spacing of the second plurality of nozzles in the second configuration.

In various implementations the conveyor also includes a drying section including a dryer mounted next to the conveyor belt. In some cases the drying section is between the gaseous ozone section and an exit of the elongated enclosure. In various cases the dryer includes an air inlet configured to operatively couple with a blower and an air outlet coupled with the air inlet and positioned near the conveyor belt. In various cases the ozone conveyor also includes one or more dryer coupling ports in the enclosure configured to operatively couple with the blower. This enables the drying system to draw ozonated air from the enclosure to feed the dryer during operation.

Another aspect of the disclosure provides an ozone disinfection system. The system includes a dual zone ozone generator, a conveyor, and an exhaust system. The dual zone ozone generator includes an ozone generator that produces gaseous ozone and an ozone injector coupled to the ozone generator for producing aqueous ozone. The conveyor includes a conveyor belt and an elongated enclosure enclosing at least part of the conveyor belt. The exhaust system is coupled to the enclosure. The conveyor also includes an aqueous ozone applicator mounted within the enclosure and coupled with the ozone injector, and a gaseous ozone applicator mounted within the enclosure and coupled with the ozone generator.

Various implementations of the ozone disinfection system include one or more of the following features and/or elements. In various cases the exhaust system includes one or more exhaust ports in the elongated enclosure, a blower operatively coupled to the one or more exhaust ports, and an ozone destruct device operatively coupled to the blower. In some cases the exhaust system is configured to draw more ozonated air from the enclosure than is introduced by the gaseous ozone applicator to thereby maintain a net negative air pressure within the elongated enclosure during operation and leaks of gaseous ozone from the elongated enclosure. In some cases the exhaust system is configured to draw ozonated air from the enclosure at a rate of about 200 or more cubic feet per minute and the gaseous ozone applicator is configured to dispense gaseous ozone at a rate of about 1 cubic foot per minute. According to various implementations, the aqueous ozone applicator is mounted between an entrance of the elongated enclosure and the gaseous ozone applicator and the gaseous ozone applicator is mounted between the aqueous ozone applicator and an exit of the elongated enclosure.

In various implementations the ozone disinfection system also includes a drying system including a dryer mounted within the enclosure. In some cases the drying section is between the gaseous ozone applicator and an exit of the elongated enclosure. In various cases the elongated enclosure includes one or more dryer coupling ports operatively coupled to the dryer, thereby enabling the drying system to draw ozonated air from the enclosure to feed the dryer during operation.

Another aspect of the disclosure provides a method for disinfecting with ozone. The method includes moving a product along a conveyor belt through an elongated enclosure. The method also includes applying aqueous ozone to the product in a first portion of the elongated enclosure after the product enters the elongated enclosure and applying gaseous ozone to the product in a second portion of the elongated enclosure following the first portion. The method further includes maintaining a net negative air pressure within the elongated enclosure by exhausting ozonated air from the elongated enclosure more quickly than introducing gaseous ozone when applying the gaseous ozone to the product. In various implementations the method includes drawing ozonated air from the enclosure and blowing the ozonated air on the product in a drying section of the enclosure following the second portion and before an exit of the elongated enclosure.

### BRIEF DESCRIPTION OF THE DRAWINGS

FIG. 1 is a process flow diagram for an ozone disinfection system according to various implementations.

FIG. 2 is a side view of an ozone conveyor according to various implementations.

FIGS. 3-4 are top and end views, respectively, of the ozone conveyor of FIG. 2.

FIGS. 5-6 are first and second perspective views, respectively, of the ozone conveyor of FIG. 2.

FIG. 7 is a detailed view A of a gaseous section of the ozone conveyor shown in FIG. 6.

FIG. 8 is a detailed view B of an aqueous section of the ozone conveyor shown in FIG. 6.

FIG. 9 is a perspective view of a blower assembly according to various implementations.

FIG. 10 is a front view of a dual zone ozone generator according to various implementations.

FIG. 11 is a process flow diagram for an ozone generator according to various implementations.

FIG. 12 is a process flow diagram for an ozone injector according to various implementations.

FIGS. 13-16 are front, side, top and perspective views, respectively, of a water chiller according to various implementations.

FIG. 17 is a perspective view of another ozone conveyor according to various implementations.

### DETAILED DESCRIPTION

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Ozone, a gas that is a triatomic form of oxygen, has been used for years in applications such as treatment of municipal water and bottled water. Ozone has enjoyed a long history of use and is known as a broad-spectrum biocide against viruses, bacteria, biofilms, fungi and protozoa—none of which can build up a resistive tolerance to ozone because ozone disinfects by oxidation processes. Ozone does not act as a systemic poison to microorganisms, but rather, destroys them by oxidation consequently making it impossible for a microorganism to build up any resistance to oxidation.

An ozone conveyor is provided according to one aspect of the disclosed technology. The ozone conveyor applies both aqueous ozone and gaseous ozone to a product moving on the conveyor. In various implementations the conveyor includes an elongated enclosure with an entrance at a first end and an exit at a second end of the enclosure. The conveyor also includes a conveyor belt that extends through the enclosure from the entrance to the exit. The ozone conveyor further includes an aqueous ozone section and a gaseous ozone section. The aqueous ozone section includes an aqueous ozone applicator mounted next to the conveyor belt for applying aqueous ozone to product on the belt. The gaseous ozone section includes a gaseous ozone applicator mounted next to the conveyor belt for applying gaseous ozone to product on the belt. The ozone conveyor also includes one or more exhaust ports configured to connect to an exhaust system for removing gaseous ozone from the enclosure. In various implementations the conveyor also includes a drying section with one or more dryers that dry the product on the conveyor belt using, for example, recycled ozonated air from the conveyor.

According to another aspect of the disclosure, an ozone disinfection system is provided. The ozone disinfection system includes a dual zone ozone generator, a conveyor, and an exhaust system. The dual zone ozone generator includes an ozone generator that produces gaseous ozone and an ozone injector that produces aqueous ozone. The conveyor includes an elongated enclosure that encloses at least part of the conveyor belt and an aqueous ozone applicator and a gaseous ozone applicator mounted within the enclosure. The aqueous and gaseous ozone applicators apply aqueous ozone and gaseous ozone, respectively, to product moving along the belt through the enclosure. The exhaust system is connected to and creates a negative air pressure within the enclosure. The negative pressure draws ozonated air from the conveyor, thus reducing the likelihood that gaseous ozone will leak from other areas of the conveyor. In various cases the system also includes a drying system that draws ozonated air from the conveyor enclosure and blows the ozonated air onto product for drying and/or additional disinfection.

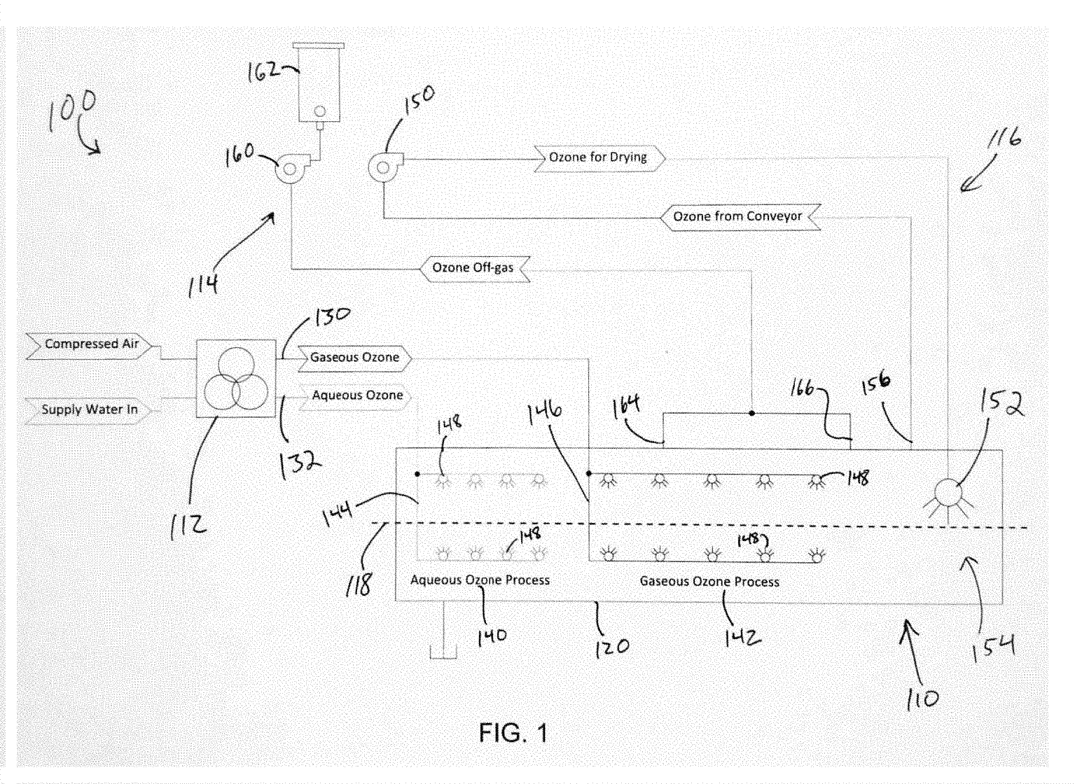

FIG. 1 is a process flow diagram of an ozone disinfection system 100 according to various implementations of the disclosed technology. In the depicted example, the disinfection system 100 includes, among other things, a conveyor 110 operatively coupled with a dual zone ozone generator 112, an exhaust system 114, and a drying system 116. The conveyor includes a conveyor belt 118 that moves product through an enclosure 120 of the conveyor.

The dual zone ozone generator 112 is configured to manufacture ozone from dry compressed air. According to various implementations, the generator first filters and regulates the incoming air before feeding it to an oxygen concentrator, which removes nitrogen from the air. The resulting oxygen stream is fed to one or more ozone generator cells. A manifold distributes the ozone gas produced by the cells to a first generator outlet 130 and an injection port. As shown in FIG. 1, the first generator outlet 130 provides a source of gaseous ozone for the conveyor 110. The gaseous ozone directed to the injection port is injected into water through a venturi injector. The ozonated water is then mixed and collected in a contact tank and provided to the conveyor through a second generator outlet 132.

As shown in FIG. 1, the conveyor 110 includes an aqueous ozone section 140 and a gaseous ozone section 142. An aqueous ozone applicator 144 mounted within the aqueous ozone section is coupled with the second generator outlet 132 of the dual zone ozone generator 112. The first generator outlet 130 of the dual zone ozone generator is coupled with a gaseous ozone applicator 146 mounted within the gaseous ozone section 142 of the conveyor. In various implementations the aqueous and/or gaseous ozone applicators include one or more spray nozzles 148. In various implementations each ozone applicator includes multiple spray nozzles 148 connected together and spaced apart next to the conveyor belt 118. As shown in the example of FIG. 1, in some cases the aqueous and/or gaseous ozone applicators include spray nozzles 148 positioned both above and below the conveyor belt 118. In some cases the aqueous and/or gaseous ozone applicators may have spray nozzles only mounted above the conveyor belt or below the belt depending upon the disinfection needs for particular implementations.

According to various implementations, the drying system 116 includes a fan or blower 150 that blows air onto product moving along the conveyor belt 118. In some cases the drying system 116 may include two or more fans or blowers. As depicted in FIG. 1, in various implementations the drying system includes a blower 150 that is connected to an air outlet or manifold 152 positioned near the conveyor belt within a drying section 154 of the conveyor enclosure 120. In some cases the air outlet 152 is an air knife. In various applications one or more blowers are connected to a first air outlet mounted over the conveyor belt and a second air outlet is mounted underneath the conveyor belt. As an example, in some implementations the drying system 116 includes a first air knife mounted over the belt and a second air knife mounted below the belt.

In some cases the drying system 116 may draw fresh air from outside the enclosure 120 for drying the product. As shown in FIG. 1, in various implementations the drying system 116 draws air containing gaseous ozone from the conveyor enclosure 120 and recirculates the ozonated air to feed the air outlet 152 mounted within the drying section 154 of the conveyor. FIG. 1 depicts an example in which the conveyor enclosure 120 includes an air outlet 156 coupled with the blower 150, which in turn is coupled with the air outlet (e.g., air knife) 152 mounted above the conveyor belt in the drying section of the enclosure. This configuration thus dries the product moving along the belt while also providing additional disinfection with any residual ozone in the recirculated air.

According to various implementations, the exhaust system 114 for the ozone disinfection system 110 is configured to remove ozonated air from the conveyor enclosure 120 and remove gaseous ozone from the air before safely venting the exhausted air. As shown in FIG. 1, in various cases the exhaust system 116 includes a fan or blower 160 that is coupled between the conveyor enclosure 120 and an ozone destruct device 162. FIG. 1 shows that the enclosure 120 includes two exhaust ports connected to the blower 160, though one, two, or more exhaust ports may be included. In various implementations the exhaust system 116 includes a first exhaust port 164 located generally near the beginning of the gaseous ozone section 142 and a second exhaust port 166 located generally near the end of the gaseous ozone section 142.

In various implementations the gaseous ozone applicator 146 and the ozone exhaust system 114 are configured so that a net negative air pressure is maintained within the conveyor enclosure 120. As an example, the blower 160 of the exhaust system may be sized and set to draw more air from the enclosure 120 than the gaseous ozone applicator 146 introduces to the enclosure. Thus, the exhaust system 116 is configured to more than compensate for the positive flow of gaseous ozone into the enclosure from the gaseous ozone applicator. The resulting net negative air pressure reduces the likelihood that gaseous ozone will leak from the conveyor 110 and pose a safety hazard to people in the area around the enclosure 120. In some implementations the disinfection system 110 further includes one or more ozone monitors that detect and measure gaseous ozone in the atmosphere as a further safety precaution.

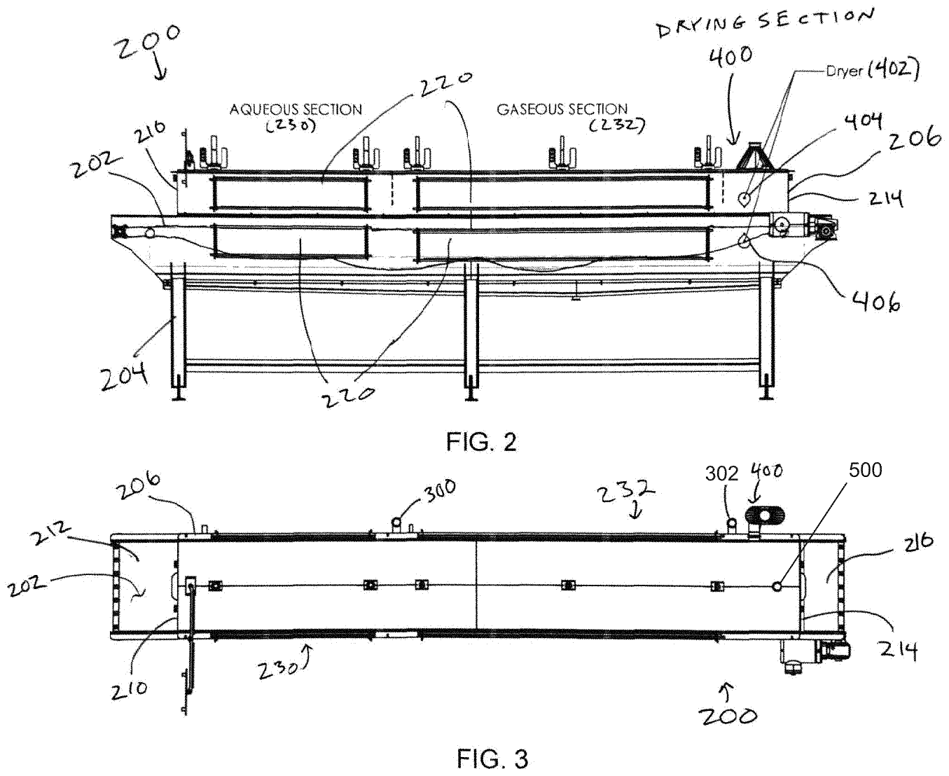

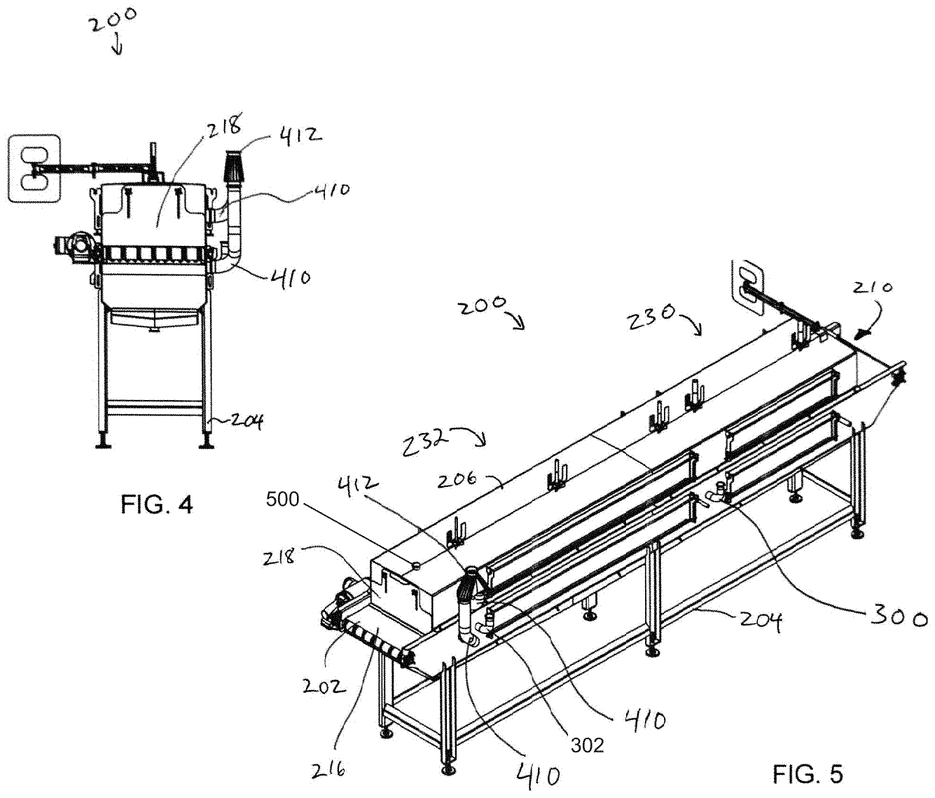

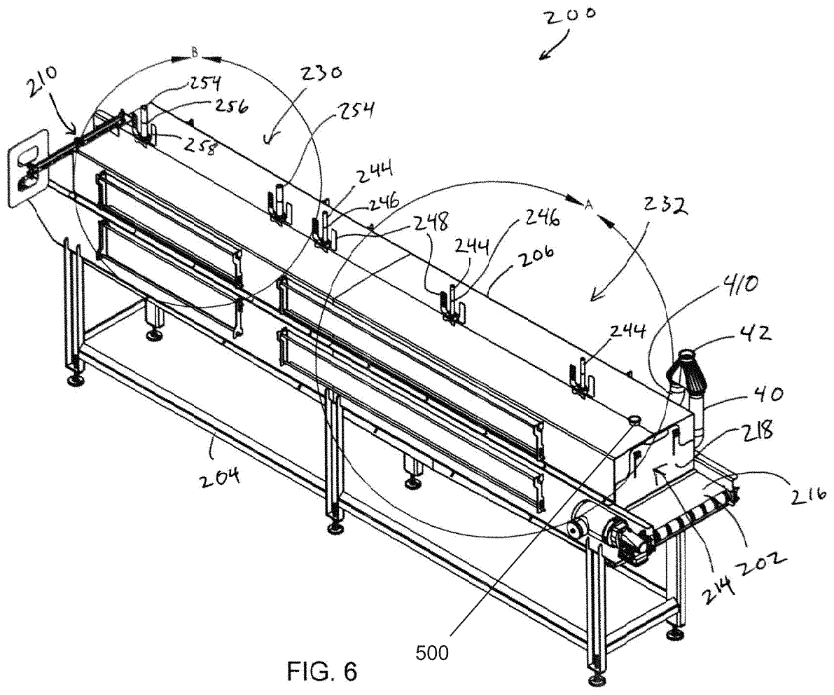

FIGS. 2-8 are various views of an ozone conveyor 200 according to various implementations of the disclosed technology. More specifically, FIG. 2 provides a side view of the ozone conveyor 200, while FIGS. 3-4 provide top and end views and FIGS. 5-6 provide first and second perspective views, respectively. The conveyor 200 generally includes a conveyor belt 202 supported by rollers and a drive motor, which are in turn mounted to a frame 204. The conveyor belt 202 extends through an enclosure 206, also mounted to the frame 204. An outline of the belt 202 is shown superimposed upon the side view of the ozone conveyor 200 in FIG. 2 to indicate the belt's position within the enclosure 206.

According to various implementations, an ozone conveyor includes a conveyor enclosure that provides enclosed spaces both above and below the conveyor belt to accommodate, for example, ozone applicators positioned both above and below the belt. It is contemplated that in various implementations an enclosure may only enclose the belt along with ozone applicator(s) positioned only above or only below the belt.

As shown in the implementation depicted in FIGS. 2-8, the conveyor enclosure 206 extends both above and below the conveyor belt 202 along nearly the entire length of the belt. In the depicted example, an entrance 210 of the enclosure 206 is positioned near an exposed first end 212 of the belt while the enclosure's exit 214 is positioned near an exposed second end 216 of the belt. In various implementations the entrance and exit include an opening covered by a movable flap 218, which serves to generally close off the ends of the enclosure while also allowing product on the conveyor belt 202 to enter and exit the enclosure. In some cases the enclosure's entrance 210 and exit 214 are about three to four inches tall. As shown in the example in the drawings, in various implementations the enclosure 206 includes doors 220 which provide access to the interior of the enclosure for adjusting ozone applicators and cleaning the conveyor 200.

The ozone conveyor 200 includes an aqueous ozone section 230 and a gaseous ozone section 232. As shown in FIGS. 2-8, the aqueous ozone section 230 includes a first portion of the conveyor enclosure 206 and the gaseous ozone section 232 includes a second portion of the conveyor enclosure 206. In various implementations the aqueous ozone section 230 is closer to the enclosure entrance 210 and the first end 212 of the conveyor belt than the gaseous ozone section 232. This relative arrangement allows for aqueous ozone to be applied to product traveling on the belt before gaseous ozone is applied to the product. In various cases the aqueous ozone can thus rinse various substances (e.g., marinades, spices, salts, sugars, and other coatings) off the product while also disinfecting the product. The subsequent application of gaseous ozone further disinfects the product and, in some cases, may also dry the product to some degree.

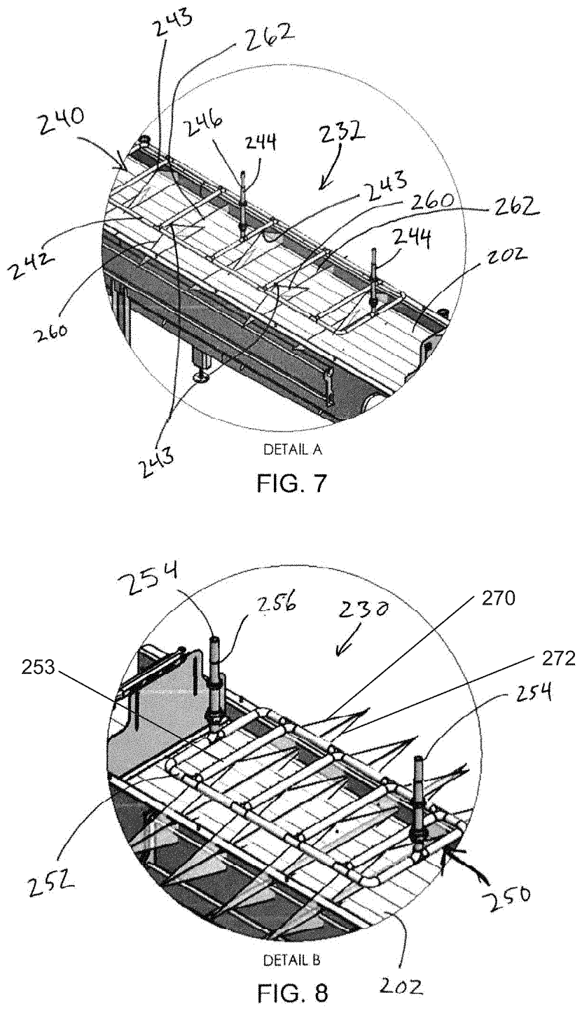

FIG. 7 is a detailed view A of the gaseous section 232 of the ozone conveyor 200 of FIG. 6. The gaseous section is shown with part of the enclosure 206 removed to illustrate one possible example of a gaseous ozone applicator 240 mounted next to the conveyor belt 202. In various implementations the gaseous ozone applicator 240 includes a spray manifold 242 and multiple nozzles 243 positioned on crossbars extending across the manifold. The applicator further includes pipes 244 connected to the spray manifold that extend through and are mounted to the top of the enclosure 206 as shown in FIG. 6. According to various implementations, a conduit (e.g., a hose or pipe) leading from a source of gaseous ozone is connected to one or more of the pipes 244 in order to provide a flow of gaseous ozone to the spray manifold 242 and nozzles 243.

According to various implementations the vertical position of the gaseous ozone applicator 240 can be adjusted by securing the pipes 244 to the enclosure in different positions. In some cases the position of the gaseous applicator is set by aligning markings 246 on the pipes 244 with measurement guides 248 extending from the top of the enclosure 206. In various cases the gaseous ozone applicator 240 can be adjustably mounted so that the spray manifold and nozzles are within a range of about three to six inches above the conveyor belt 202.

The nozzles 243 attached to the crossbars of the spray manifold 242 dispense gaseous ozone down upon the conveyor belt 202 to disinfect product traveling on the belt. According to various implementations, an additional gaseous ozone applicator mounted underneath the conveyor belt 202 dispenses gaseous ozone up toward the product from below the belt. In these cases the conveyor belt 202 has an open design such as, for example, a wire or wire-mesh belt that allows the gaseous ozone to pass through from below. In some cases the additional gaseous ozone applicator is fixedly mounted beneath the belt while another applicator is adjustably mounted above the belt as discussed above. In various implementations the additional gaseous ozone applicator mounted below the belt is fluidly connected with the gaseous ozone applicator 240 mounted above the conveyor belt and/or to the mounting and supply pipes 244 in order to supply the lower applicator with gaseous ozone.

FIG. 8 is a detailed view B of the aqueous section 230 of the ozone conveyor 200 of FIG. 6. The aqueous section is shown with part of the enclosure 206 removed to illustrate one possible example of an aqueous ozone applicator 250 mounted next to the conveyor belt 202. In various implementations the aqueous ozone applicator 250 includes a spray manifold 252 and multiple nozzles positioned on crossbars 253 extending across the manifold 252. The applicator 250 further includes pipes 254 connected to the spray manifold that extend through and are mounted to the top of the enclosure 206 as shown in FIG. 6. According to various implementations, a conduit (e.g., a hose or pipe) leading from a source of aqueous ozone is connected to one or more of the pipes 254 in order to provide a flow of aqueous ozone to the crossbars 253 and nozzles of the spray manifold 252.

According to various implementations, the position of the aqueous ozone applicator 250 can be vertically adjusted above the conveyor belt in the same manner described above with respect to the gaseous ozone applicator 240. As shown in FIGS. 6 and 8, in various cases the pipes 254 extending out from the enclosure 206 contain markings 256 for aligning with measurement guides 258 in order to mount the aqueous ozone applicator 250 in a preset position. Further, in various cases an additional aqueous ozone applicator is mounted underneath the conveyor belt 202 to dispense aqueous ozone upward in a manner similar to the upward-dispensing gaseous ozone applicator. In this way product traveling on the belt can be disinfected and rinsed with aqueous ozone from both above and below. In various implementations the additional aqueous ozone applicator mounted below the belt is fluidly connected with the aqueous ozone applicator 250 mounted above the conveyor belt and/or to the mounting and supply pipes 254 in order to supply the lower applicator with aqueous ozone.

As can be appreciated from the drawings, FIGS. 7 and 8 depict two different examples of ozone applicators. Referring to FIG. 7, the gaseous ozone applicator 240 includes a spray manifold 242 containing nozzles 243 that alternate positions across the width of the belt as the spray manifold extends along the length of the belt. The approximate spray pattern 260 is illustrated for each nozzle. In various cases this arrangement provides less overlap between nozzles and allows for the use of a relatively higher spray rate, while the gaps between nozzles can reduce splashing. As an example, in various cases each nozzle 243 of the gaseous ozone applicator 240 sprays gaseous ozone at a rate of 1-2 liters per minute, at a pressure of about 15 psi. In various implementations the lower gaseous ozone applicator mounted below the belt (not shown in FIG. 7) has a similar or identical configuration as the upper gaseous applicator 240. In the example of FIG. 7, the lower gaseous ozone applicator is positioned under the belt 202 so that spray patterns 262 from its nozzles alternate with the spray patterns 262 from the nozzles 243 of the upper applicator 240. The combination of alternating upper and lower spray nozzles thus provides greater coverage for the disinfecting gaseous ozone dispensed by the nozzles.

Turning to FIG. 8, the aqueous ozone applicator 250 in this example includes a spray manifold 252 with more densely positioned nozzles than the example in FIG. 7. In the example of FIG. 8, the spray manifold 252 includes pairs of nozzles positioned on the crossbars 253 that are spaced along the length of the applicator 250. In some cases the nozzles on a crossbar are spaced evenly across the width of the spray manifold 252 splitting the length of the crossbar in thirds. The approximate spray pattern 270 is illustrated for each nozzle. The gap between successive pairs of nozzles can reduce splash while the pairing of nozzles provides more spray overlap. In various implementations this configuration also allows the use of a relatively lower spray rate. As an example, in various cases each nozzle of the aqueous ozone applicator 250 sprays aqueous ozone at a rate of 0.5-1.0 gal/min at a pressure of about 30 psi. In various implementations a lower aqueous ozone applicator mounted below the belt (not shown in FIG. 8) has a similar or identical configuration as the upper gaseous applicator 250. In the example of FIG. 8, the lower aqueous ozone applicator is positioned under the belt 202 so that spray patterns 272 from its nozzles align with the spray patterns 270 from the nozzles of the upper applicator 250. In some cases the combination of aligned upper and lower spray nozzles ensures coverage for the disinfecting aqueous ozone dispensed by the nozzles at a relatively lower rate.

According to various implementations the configurations of aqueous and gaseous ozone applicators may be the same or different. The implementation shown in FIGS. 7-8 illustrates an example in which the gaseous ozone applicator 240 (along with the gaseous applicator under the belt) has a less dense nozzle configuration with a relatively higher spray rate while the aqueous ozone applicator 250 (along with the aqueous applicator under the belt) has a denser nozzle configuration with a lower spray rate. Each gaseous applicator in this example has five nozzles for a total of ten nozzles in the gaseous ozone section. Each aqueous applicator has eight nozzles for a total of sixteen nozzles in the aqueous ozone section. In various implementations both aqueous and gaseous ozone applicators can have a lower density of nozzles such as in FIG. 7 and in various other implementations both aqueous and gaseous ozone applicators can have a denser nozzle configuration such as in FIG. 8. For example, in some cases the gaseous ozone applicators are configured as in FIG. 7 and the aqueous ozone applicators each have four alternating nozzles like in FIG. 7 for a total of eight nozzles. As another example, in some cases the aqueous ozone applicators are configured as in FIG. 8 and the gaseous ozone applicators each have ten nozzles in pairs like in FIG. 8 for a total of twenty nozzles. In another example the gaseous ozone applicators can be configured in a less dense arrangement such as in FIG. 8 while the aqueous ozone applicators can be configured in a denser arrangement such as in FIG. 7.

According to various implementations, the ozone conveyor 200 is configured so that a negative air pressure can be maintained within the enclosure 206 to reduce the risk that gaseous ozone will escape from the conveyor 200 into the surrounding environment. In such implementations the ozone conveyor 200 includes at least one exhaust port. As shown in FIGS. 3 and 5, in various cases the ozone conveyor 200 includes two exhaust ports 300, 302 in communication with the interior of the enclosure 206. In some cases the conveyor may include one or more than two exhaust ports. The exhaust ports 300, 302 can be any suitable pipe or hose that is coupled to the interior of the enclosure 206. During operation, the exhaust ports 300, 302 are coupled with an exhaust system that draws air from the enclosure 206 to maintain a negative pressure within the ozone conveyor 200.

According to various implementations the movable flaps 218 covering the entrance and exit of the enclosure 206 help maintain the negative pressure. In various implementations connection points of the enclosure 206 are further sealed with a food safe and ozone resistant sealant. In addition, in some cases the enclosure doors 220 and/or door frames on the enclosure can include seals or gaskets to prevent ozone from leaking outside the enclosure. In some implementations the vacuum generated by the exhaust system may be sufficiently strong to overcome any leaks presented by connection points, the doors and/or the flaps, thus potentially reducing the need for additional sealing measures. Various implementations of the conveyor 200 further include one or more ozone monitors that detect and measure gaseous ozone in the surrounding atmosphere as a further safety precaution.

Returning to FIG. 2, in various implementations the ozone conveyor 200 includes a drying section 400. As depicted, the drying section 400 in this example includes a dryer 402 positioned after the gaseous ozone section 232. The dryer 402 is part of a drying system that includes, among other things, a fan or blower coupled to the dryer 402 that blows air onto product moving along the conveyor belt 202. In various implementations the dryer 402 includes an air outlet or manifold positioned near the conveyor belt. In some cases the air outlet is an air knife. In the example shown in FIGS. 2-8, the dryer 402 includes a first air outlet 404 mounted over the conveyor belt 202 and a second air outlet 406 mounted underneath the conveyor belt. In some implementations the first air outlet 404 includes a first air knife mounted over the belt and the second air outlet 406 includes a second air knife mounted below the belt 202. The first and second air outlets 404, 406 are fed by air inlets 410 that are coupled to the air outlets through the enclosure wall. In various implementations the air inlets 410 can be any suitable pipe or hose capable of coupling the air outlets 404, 406 to a source of drying air. As shown in FIGS. 4-6, the air inlets 410 in this example are combined at a manifold 412 that can then be connected to a source of drying air.

In some cases the dryer 402 may be fed with air drawn from outside the enclosure. As shown in FIGS. 3 and 5, in various implementations the ozone conveyor 200 includes at least one dryer coupling 500 that allows the drying system to draw air containing gaseous ozone from within the conveyor enclosure 202 and recirculate the ozonated air to feed the dryer 402. This configuration thus enables the dryer 402 to dry the product moving along the belt while also disinfecting the product with any residual ozone in the recirculated air.

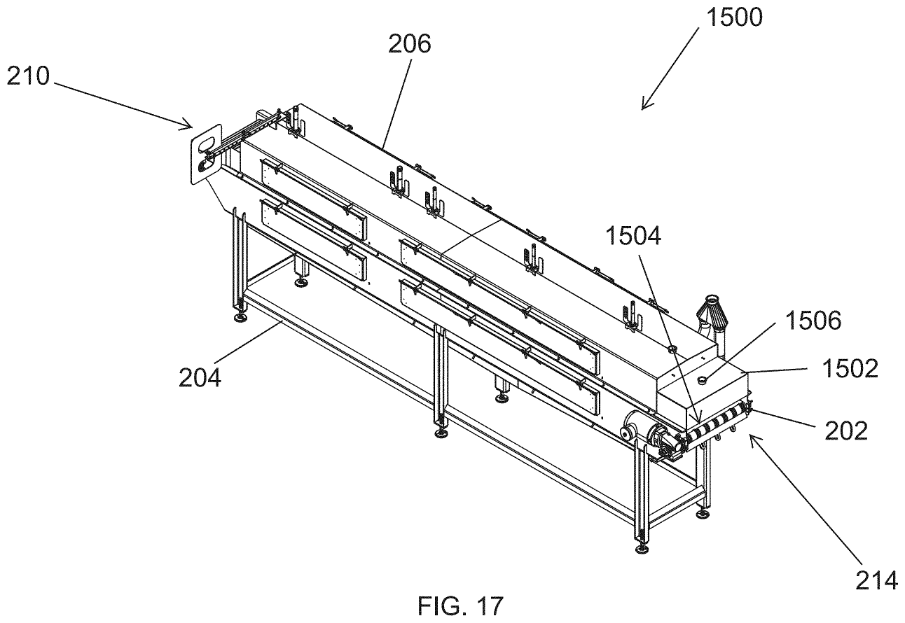

Turning now to FIG. 17, a perspective view of another ozone conveyor 1500 according to various implementations of the disclosed technology. The ozone conveyor 1500 in this example is similar in many respects to the ozone conveyor 200 shown in FIG. 2-8 and like parts are indicated by the same reference numbers. As shown in FIG. 17, in various cases the ozone conveyor 1500 includes an additional exhaust hood 1502, which is mounted at the exit 214 of the conveyor's enclosure 206. The exhaust hood 1502 extends down on each side of the conveyor belt 202 and also extends down across the conveyor belt to a predetermined height that leaves an opening or slot 1504 for product to exit the conveyor. In some cases, for example, the predetermined height is based on the dimensions of the product being disinfected (e.g., in some cases 4 inches).

The exhaust hood 1502 includes an exhaust port 1506. The exhaust port 1506 can be any suitable pipe, hose, or opening that is coupled to the interior of the hood 1502. During operation, the exhaust port 1506 is coupled to an exhaust system along with the other exhaust ports 300, 302 to draw ozonated air from the enclosure 206 and exhaust hood 1502. Such a system can help maintain a negative pressure within the ozone conveyor 200 and reduce and/or prevent leaking of gaseous ozone from the conveyor to the surrounding area.

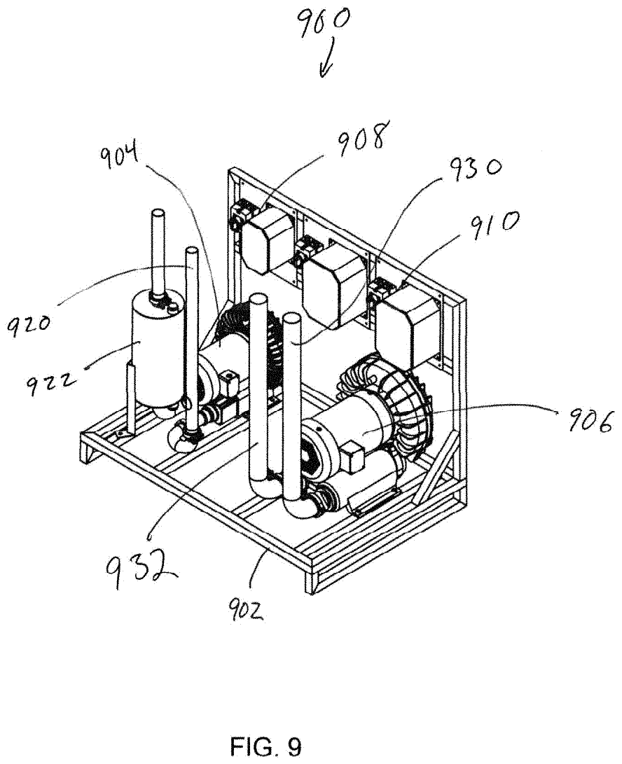

Returning to FIG. 9 is a perspective view of a blower assembly 900 according to various implementations of the disclosed technology. The blower assembly 900 includes a frame 902 on which is mounted a first blower 904 and a second blower 906. In various implementations the first blower 904 is part of an exhaust system capable of coupling with one or more exhaust ports of an ozone conveyor. In some cases the second blower 906 is part of a drying system capable of coupling with a dryer of an ozone conveyor. Control electronics 908, 910 are also mounted to the frame for powering and operating the first and second blowers 904, 906, respectively. In various implementations, the frame 902 is a loading platform (e.g., a skid) that can be moved and repositioned by machinery (e.g., a skid loader). Accordingly, the blower assembly 900 provides a compact and convenient form factor for installing an exhaust system and drying system as part of an ozone conveyor and ozone disinfection system.

According to various implementations, the first blower 904 is coupled with an inlet pipe 920 that is configured to couple with the exhaust port(s) of an ozone conveyor. As an example, the inlet pipe 920 can be fluidly coupled to the exhaust port 300 of the ozone conveyor 200 shown in FIG. 3. The first blower 904 is also coupled with an ozone destruct unit 922 that removes gaseous ozone from the air drawn from the ozone conveyor prior to releasing the air to the atmosphere. The second blower 906 is coupled with an inlet pipe 930 and an outlet pipe 932 that allow the blower 906 to be connected to a source of drying air and/or an ozone conveyor dryer. As an example, the inlet pipe 930 can be coupled to the dryer coupling 500 of the ozone conveyor 200 as shown in FIG. 3. Further, the outlet pipe 932 can be coupled to the dryer manifold 412 of the ozone conveyor in order to feed ozonated air from the conveyor's enclosure to the conveyor's dryer 402. It is also contemplated that the inlet pipe 930 could in some cases be connected to another source of drying air that does not contain ozone. One example includes air drawn from outside the enclosure and/or fresh air drawn from outside of a building.

In various implementations the first and second blowers 904, 906 are selected so that they are capable of producing the desired vacuum or air flow. As an example, in some cases the gaseous ozone applicator(s) within an ozone conveyor dispense gaseous ozone at a rate of 30 liters/minute (e.g., approximately 1 cfm) while the first blower 904 draws air from the conveyor enclosure at a rate of 200-300 cfm. Accordingly the relatively large flow rate of the first blower 904 compared to the gaseous ozone spray rate creates a negative pressure within the enclosure that greatly increases the likelihood that most, if not all, gaseous ozone will be drawn out by the first blower rather than leaking from other points of the ozone conveyor. In various implementations the maximum flow rate and/or vacuum rating of the second blower 906 is selected based on the desired air output for the ozone conveyor's dryer. In some cases the second blower 906 has a flow rate of about 400 cfm.

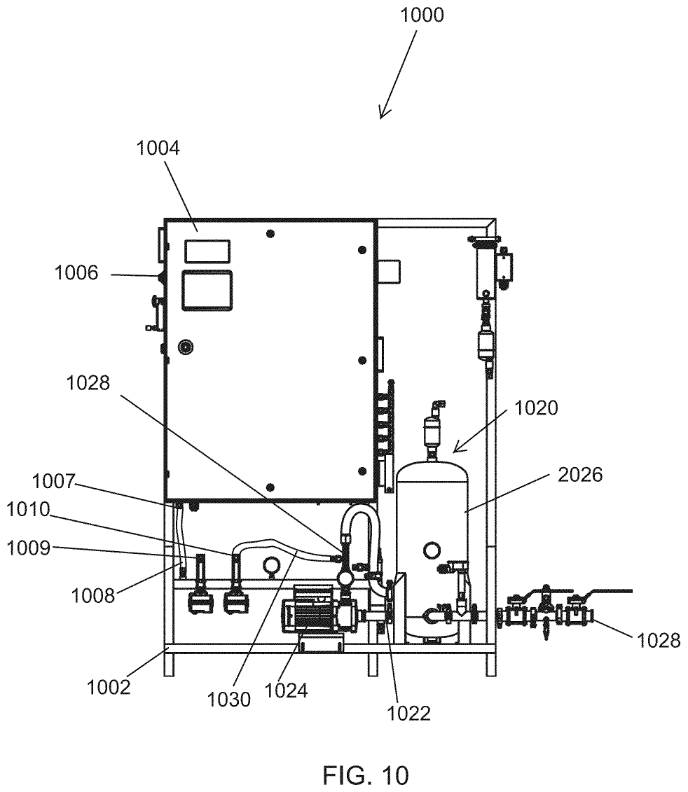

FIG. 10 is a front view of a dual zone ozone generator 1000 according to various implementations. Generally speaking, the dual zone ozone generator 1000 is capable of generating output streams of gaseous ozone and aqueous ozone for disinfecting gaseous and aqueous ozone sections of an ozone conveyor in various implementations. In some cases the dual zone ozone generator 1000 is one possible implementation of the dual zone ozone generator 112 illustrated as part of the ozone disinfection system 100 shown in FIG. 1.

Referring again to FIG. 10, the dual zone generator 1000 includes a frame 1002 upon which is mounted a gaseous ozone generator 1004. In various implementations, the frame 1002 is a loading platform (e.g., a skid) that can be moved and repositioned by machinery (e.g., a skid loader). Accordingly, the dual zone generator 1000 has a compact and convenient form factor for installing as part of an ozone conveyor and ozone disinfection system. In various implementations the gaseous ozone generator 1004 has an input 1006 for receiving dry, compressed air. For example, an on-site source of compressed air may be coupled to the generator input 1006. In various cases an air compressor mounted to the frame 1002 supplies the generator 1004 with compressed air.

According to its general operation, the gaseous ozone generator 1004 conditions and feeds the compressed air to one or more ozone generating cells. The ozone generated by the cells is then fed to an outlet 1007 for use. As shown in FIG. 10, in various implementations a hose 1008 couples the generator outlet 1007 to a distribution manifold that includes a first gaseous outlet 1009 and a second gaseous outlet 1010. The first gaseous outlet 1009 can be used to feed gaseous ozone to one or more gaseous ozone applicators in an ozone conveyor and disinfection system. The second gaseous outlet is coupled to an ozone injection system for making aqueous ozone.

As shown in FIG. 10, the dual zone generator 1000 also includes an ozone injection system 1020. The injection system 1020 includes a water inlet 1022, a pump 1024, a contact tank 1026, a venturi inlet 1028 coupled between the pump 1024 and the tank 1026, and an aqueous ozone outlet 1028. A hose 1030 connects the second gaseous outlet 1010 of the ozone generator to the venturi inlet 1028. As the pump 1024 moves water from the water inlet 1022 to the tank 1026, gaseous ozone is injected into the water by the venturi injector 1028. After mixing in the contact tank 1026, the aqueous ozone can be supplied to the aqueous ozone applicator(s) of an ozone conveyor.

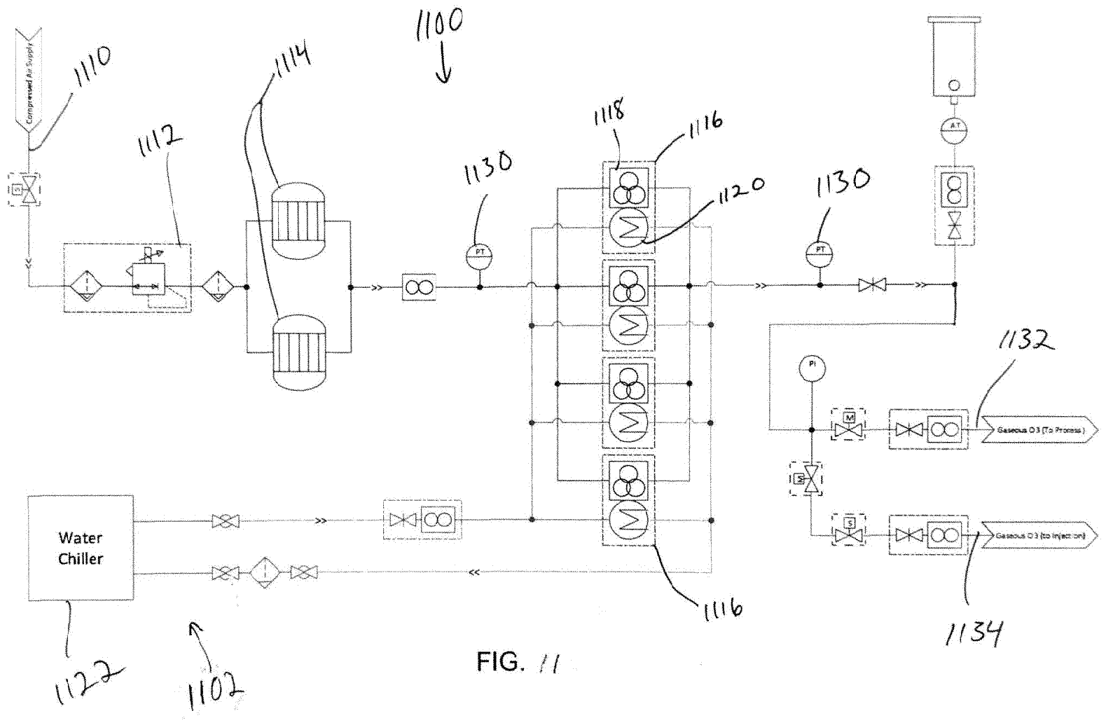

FIG. 11 is a process flow diagram for an ozone generator 1100 including a water chilling system 1102 according to various implementations. In some cases the ozone generator 1100 can be included as part of a dual zone ozone generator, such as the dual zone ozone generator 1000 depicted in FIG. 10. As shown in FIG. 11, the ozone generator 1100 has a compressed air inlet 1110. A conditioner 1112 including, for example, oil and/or particulate filters and a regulator is coupled downstream of the inlet. After being conditioned, the compressed and conditioned air is fed to one or more oxygen concentrators 1114. The oxygen concentrator(s) 1114 generate an oxygen-rich air stream that in some implementations has at least 93% oxygen.

Continuing with reference to FIG. 11, the ozone generator 1100 includes multiple ozone generation cells 1116 coupled downstream of the oxygen concentrator(s). In various implementations each ozone generation cell 1116 includes an ozone cell 1118 and a heat exchanger 1120. In various cases each of the heat exchanger 1120 is water-cooled. As shown in the example of FIG. 11, each heat exchanger 1120 is fluidly coupled with a water chiller 1122 that conditions the water being fed to the heat exchangers. In the depicted implementation the water chiller 1122, heat exchangers 1120 and a number of valves form the water chilling system 1102. The ozone generator 1100 in this example also includes a pair of pressure regulators 1130, one before and one after the ozone generation cells 1116.

As shown in FIG. 11, the ozonated air exiting the ozone generation cells is then selectively fed to two outlets. A first gaseous ozone outlet 1132 is available for outputting gaseous ozone for direct use, such as in one or more gaseous ozone applicators of an ozone conveyor. In some implementations the first gaseous ozone outlet 1132 is an example of the first gaseous outlet 1008 described above with respect to FIG. 10. Returning to FIG. 11, a second gaseous ozone outlet 1134 makes gaseous ozone available to an ozone injection system such as, for example, the injection system 1020 described above with respect to FIG. 10.

FIG. 12 is a process flow diagram for an ozone injector 1200 according to various implementations. In some cases the ozone injector 1200 is an example of the ozone injection system 1020 depicted in FIG. 10. As shown in FIG. 12, the injector 1200 in this example includes a first input 1202 for gaseous ozone. A second input 1204 of the injector intakes supply water. The supply water is fed to a contact tank 1206 and then drawn into an injection loop 1208 by a pump 1210. The injection loop 1208 includes a venturi injector 1212 also coupled with the gaseous ozone input 1202. Gaseous ozone is injected into the water as it passes through the injector 1212 and the ozonated water is then fed back into the contact tank 1206 for mixing.

Once mixed, the ozonated water is fed to an aqueous ozone outlet 1214. The aqueous ozone is thus made available for one or more aqueous ozone applicators of an ozone conveyor such as described elsewhere herein. In various implementations an ozone destruct unit 1216 is coupled to the contact tank for off-gassing and/or an aqueous ozone test port 1218 is provided for testing the ozone concentration of the output water.

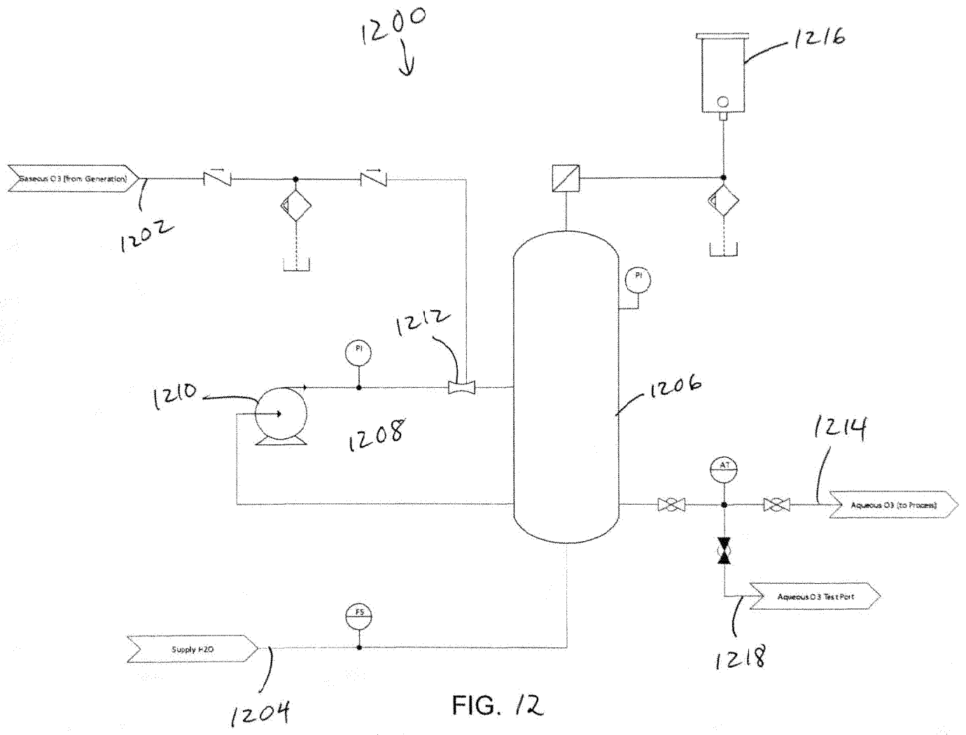

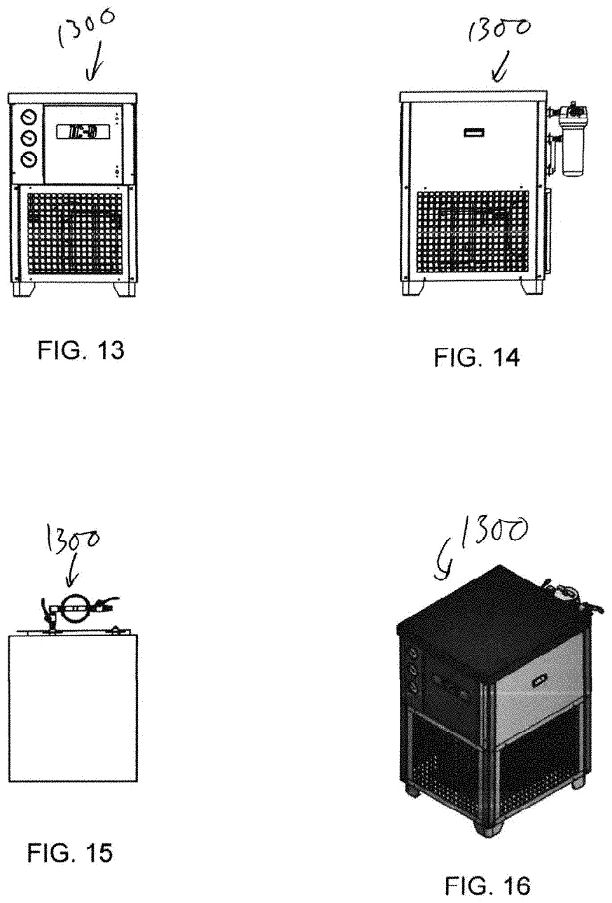

As discussed above, in various implementations the ozone generation cells of an ozone generator include heat exchangers for cooling the ozone elements. In some cases the heat exchangers are water-cooled and fed by a cooling line supplied by a water chiller. FIGS. 13-16 are front, side, top and perspective views, respectively, of one example of a water chiller 1300 according to various possible implementations of the disclosed technology.

As will be appreciated by a person skilled in the art, implementations of the disclosed technology may be utilized to disinfect a variety of products traveling along a conveyor. Various implementations include methods for disinfecting products with ozone. According to some implementations, a method for disinfecting with ozone includes moving a product along a conveyor belt through an elongated enclosure. The method further includes applying aqueous ozone to the product in a first portion of the elongated enclosure after the product enters the elongated enclosure and applying gaseous ozone to the product in a second portion of the elongated enclosure following the first portion. The method further includes maintaining a net negative air pressure within the elongated enclosure by exhausting ozonated air from the elongated enclosure more quickly than introducing gaseous ozone when applying the gaseous ozone to the product. In various implementations the method includes drawing ozonated air from the enclosure and blowing the ozonated air on the product in a drying section of the enclosure following the second portion and before an exit of the elongated enclosure. Various method implementations include one or more additional steps such as any of those discussed elsewhere herein, including with respect to the examples illustrated in the drawings.

Examples of products that may be disinfected with ozone include food products. Examples of food products include various meat products. In certain aspects, a meat product is chosen from poultry, beef, lamb, and pork. In further aspects, a meat product is a combination of the foregoing. According to further aspects, a meat product is poultry. In various implementations, the poultry is chicken.

Meat products can be in a variety of forms, including meat cuts and ground meat (for example, ground beef). Examples of meat cuts include primal cuts, subprimal cuts and retail cuts. Primal cuts include beef loins, pork loins, beef ribs, pork hams, and beef rounds. Subprimal cuts include beef strips, beef rib eyes, beef top sirloins, pork shoulder butts, pork center cut loins, pork sirloins, and beef bottom round flats. Retail cuts include sirloin steaks, stew meat, cube steaks, country style ribs, pork chops, blade steaks, cutlets, poultry thighs, poultry breasts, and poultry tenders.

Although the disclosure has been described with reference to preferred embodiments, persons skilled in the art will recognize that changes may be made in form and detail without departing from the spirit and scope of the disclosed apparatus, systems and methods.

What is claimed is:

1. An ozone conveyor, comprising:
an elongated enclosure;
a conveyor belt extending through the enclosure;
an aqueous ozone section comprising an aqueous ozone applicator mounted next to the conveyor belt;
a gaseous ozone section comprising a gaseous ozone applicator mounted next to the conveyor belt; and
one or more exhaust ports configured to connect to an exhaust system for removing gaseous ozone from the enclosure.

2. The ozone conveyor of claim 1, wherein the elongated enclosure comprises an entrance at a first end of the enclosure and an exit at a second end of the enclosure, wherein the aqueous ozone section comprises a first portion of the enclosure between the entrance and the exit and the gaseous ozone section comprises a second portion of the enclosure between the first portion of the enclosure and the exit.

3. The ozone conveyor of claim 2, further comprising an exhaust hood mounted at the exit of the enclosure, wherein the exhaust hood comprises the one or more exhaust ports.

4. The ozone conveyor of claim 1, wherein the aqueous ozone applicator is a first aqueous ozone applicator mounted above the conveyor belt and wherein the aqueous ozone section further comprises a second aqueous ozone applicator mounted below the conveyor belt.

5. The ozone conveyor of claim 1, wherein the gaseous ozone applicator is a first gaseous ozone applicator mounted above the conveyor belt and wherein the gaseous ozone section further comprises a second gaseous ozone applicator mounted below the conveyor belt.

6. The ozone conveyor of claim 1, wherein the aqueous ozone applicator comprises a first plurality of nozzles coupled to a first spray manifold in a first configuration and the gaseous ozone applicator comprises a second plurality of nozzles coupled to a second spray manifold in a second configuration.

7. The ozone conveyor of claim 6, wherein the spacing of the first plurality of nozzles in the first configuration is denser than the spacing of the second plurality of nozzles in the second configuration.

8. The ozone conveyor of claim 1, further comprising a drying section comprising a dryer mounted next to the conveyor belt.

9. The ozone conveyor of claim 8, wherein the drying section is between the gaseous ozone section and an exit of the elongated enclosure.

10. The ozone conveyor of claim 9, wherein the dryer comprises an air inlet configured to operatively couple with a blower and an air outlet coupled with the air inlet and positioned near the conveyor belt.

11. The ozone conveyor of claim 10, further comprising one or more dryer coupling ports in the enclosure configured to operatively couple with the blower, thereby enabling the drying system to draw ozonated air from the enclosure to feed the dryer during operation.

12. An ozone disinfection system, comprising:
a dual zone ozone generator, comprising:
an ozone generator that produces gaseous ozone, and
an ozone injector coupled to the ozone generator;
a conveyor comprising:
a conveyor belt,
an elongated enclosure enclosing at least part of the conveyor belt,
an aqueous ozone applicator mounted within the enclosure and coupled with the ozone injector, and
a gaseous ozone applicator mounted within the enclosure and coupled with the ozone generator; and
an exhaust system coupled to the enclosure.

13. The ozone disinfection system of claim 12, wherein the exhaust system comprises one or more exhaust ports in the elongated enclosure, a blower operatively coupled to the one or more exhaust ports, and an ozone destruct device operatively coupled to the blower.

14. The ozone disinfection system of claim 12, wherein the exhaust system is configured to draw more ozonated air from the enclosure than is introduced by the gaseous ozone applicator to thereby maintain a net negative air pressure within the elongated enclosure during operation and leaks of gaseous ozone from the elongated enclosure.

15. The ozone disinfection system of claim 14, wherein the exhaust system is configured to draw ozonated air from the enclosure at a rate of about 200 or more cubic feet per minute and the gaseous ozone applicator is configured to dispense gaseous ozone at a rate of about 1 cubic foot per minute.

16. The ozone disinfection system of claim 12, wherein the aqueous ozone applicator is mounted between an entrance of the elongated enclosure and the gaseous ozone applicator and the gaseous ozone applicator is mounted between the aqueous ozone applicator and an exit of the elongated enclosure.

17. The ozone disinfection system of claim 12, further comprising a drying system comprising a dryer mounted within the enclosure.

18. The ozone disinfection system of claim 17, wherein the drying section is between the gaseous ozone applicator and an exit of the elongated enclosure.

19. The ozone disinfection system of claim 18, wherein the elongated enclosure comprises one or more dryer coupling ports operatively coupled to the dryer, thereby enabling the drying system to draw ozonated air from the enclosure to feed the dryer during operation.

20. A method for disinfecting with ozone, comprising:
moving a product along a conveyor belt through an elongated enclosure;
applying aqueous ozone to the product in a first portion of the elongated enclosure after the product enters the elongated enclosure;
applying gaseous ozone to the product in a second portion of the elongated enclosure following the first portion;
maintaining a net negative air pressure within the elongated enclosure by exhausting ozonated air from the elongated enclosure more quickly than introducing gaseous ozone when applying the gaseous ozone to the product; and
drawing ozonated air from the enclosure and blowing the ozonated air on the product in a drying section of the enclosure following the second portion and before an exit of the elongated enclosure.
